(19) United States
(12) Patent Application Publication
(10) Pub. No.: US 2022/0401609 A1
(43) Pub. Date: Dec. 22, 2022

(54) DEVICE FOR REMOVING WAX FROM A WAX BURNING/HEATING DEVICE AND METHOD THEREFOR

(71) Applicant: PAUL WYNN, LAS VEGAS, NV (US)

(72) Inventor: PAUL WYNN, LAS VEGAS, NV (US)

(21) Appl. No.: 17/745,468

(22) Filed: May 16, 2022

Related U.S. Application Data
(60) Provisional application No. 63/211,386, filed on Jun. 16, 2021.

(51) Int. Cl. A61L 9/03

(52) U.S. Cl. CPC A61L 9/03

(57) ABSTRACT

A wax removing device for a wax heater has a plate member sized to fit within a reservoir of the wax heater. A handle extends up from the plate member.

### RELATED APPLICATIONS

This patent application is related to U.S. Provisional Application No. 63/211,386 filed Jun. 16, 2021, entitled “WAX REMOVAL SYSTEM” in the name of the same inventor, and which is incorporated herein by reference in its entirety. The present patent application claims the benefit under 35 U.S.C § 119(e).

### TECHNICAL FIELD

The present application generally relates to a wax burner/heater, and more specifically, to a device to remove wax that has melted and solidified within the wax burner/heater.

### BACKGROUND

Candles have been used for years for lighting and for decorative purposes. In general, a candle may consist of a wick encased within wax. The wick may be lit for the candle to provide light. The wax encasing the wick may be formed in different configurations for a desired decorative purpose.

Candles may also be used to provide a pleasant aroma. Scented candles have enjoyed increasing popularity in recent years and may be used not only to mask unpleasant odors and provide a desirable ambiance but also for aromatherapy.

Using candles generally requires a flame and involves combustion. Consequently, burning candles present a potential fire danger, and may also disperse combustion products into the air, some of which may be toxic, such as carbon monoxide, plastics, and other noxious hydrocarbons. Because of this, many people have started using wax burners/heaters (hereinafter wax heaters). Wax heaters are devices used to heat up wax melts which are small pieces of wax that are like candles in scent and composition. Warming the wax melts may provide a pleasant aroma without the dangers of lighting a candle.

One issue when using wax heaters is that the wax melts will melt during use. The melted wax may build up over time within the wax heater and may need to be removed. Presently, there is no easy way to remove the used melted wax from the reservoir of the wax heater after and between uses. For example, current wax heater users may be forced to remove used wax by either freezing the wax in the user's freezer and removing the wax as a whole or pouring the hot wax out into the garbage. However, there are problems that may arise from the two scenarios. First, if a user freezes the used wax using a freezer, the freezer absorbs the strong chemicals used to make the wax melts. This may cause the food stored within the freezer to smell like the wax, and possibly creating a health hazard from food contaminated with the chemicals. Second, pouring the hot melted wax from the wax heater may leave drip marks on the wax heater, rendering it practically useless, and certainly unattractive to the user. The other issue lies in the fact that it is easy to burn oneself and or catch your garbage on fire using this method.

Therefore, it would be desirable to provide a device and method that overcomes the above. The device and method would allow the one to remove the used wax melt from the reservoir of the wax heater.

### SUMMARY

In accordance with one embodiment, a wax removing device for a wax heater is disclosed. The wax removing device has a plate member sized to fit within a reservoir of the wax heater. A handle extends up from the plate member.

In accordance with one embodiment, a wax removing device for a wax heater is disclosed. The wax removing device has a circular plate member sized to fit within a reservoir of the wax heater. A handle extends up from a center area of the plate member. The handle has a rod member attached to the plate member. A knob is attached to a distal end of the rod member.

### BRIEF DESCRIPTION OF THE DRAWINGS

The present application is further detailed with respect to the following drawings. These figures are not intended to limit the scope of the present application but rather illustrate certain attributes thereof. The same reference numbers will be used throughout the drawings to refer to the same or like parts.

FIG. 1 is a bottom perspective view of an exemplary embodiment of a wax removal device used in a wax heater in accordance with an embodiment of the present invention;

FIG. 2 is a perspective view of an exemplary embodiment of the wax removal device positioned within the wax heater in accordance with an embodiment of the present invention;

FIG. 3 is a perspective view of an exemplary embodiment of the wax removal device positioned within the wax heater with wax melts positioned within a reservoir of the wax heater in accordance with an embodiment of the present invention;

FIG. 4 is a perspective view of an exemplary embodiment of the wax removal device positioned within the wax heater with the wax melts melted and hardened within the reservoir of the wax heater in accordance with an embodiment of the present invention; and

FIG. 5 is a perspective view of an exemplary embodiment of the wax removal device used in the wax heater with the wax removal device lifting the melted wax melts from the reservoir of the wax heater in accordance with an embodiment of the present invention.

### DESCRIPTION OF THE APPLICATION

The description set forth below in connection with the appended drawings is intended as a description of presently preferred embodiments of the disclosure and is not intended to represent the only forms in which the present disclosure can be constructed and/or utilized. The description sets forth the functions and the sequence of steps for constructing and operating the disclosure in connection with the illustrated embodiments. It is to be understood, however, that the same or equivalent functions and sequences can be accomplished by different embodiments that are also intended to be encompassed within the spirit and scope of this disclosure.

Embodiments of the exemplary device and method disclose a device for use in a wax heater that may be able to remove used wax melts from the wax heater. The device may be placed within a pan of the wax heater and after the wax melts have been used and hardened, the device may be lifted out from the pan to remove the hardened used wax melts.

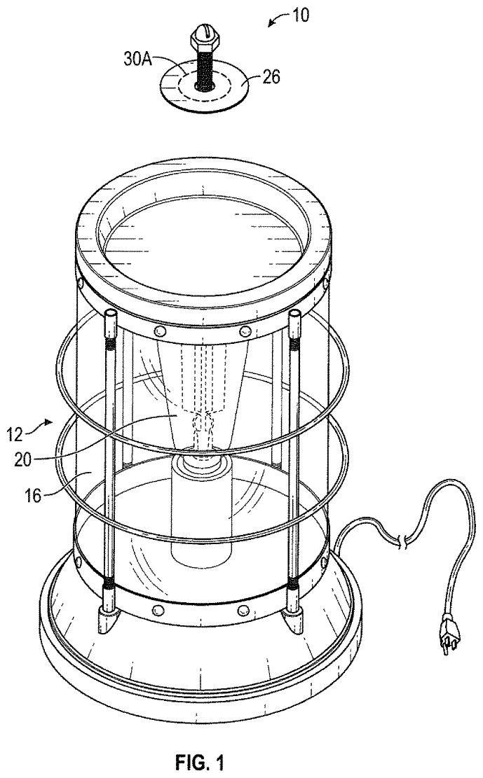

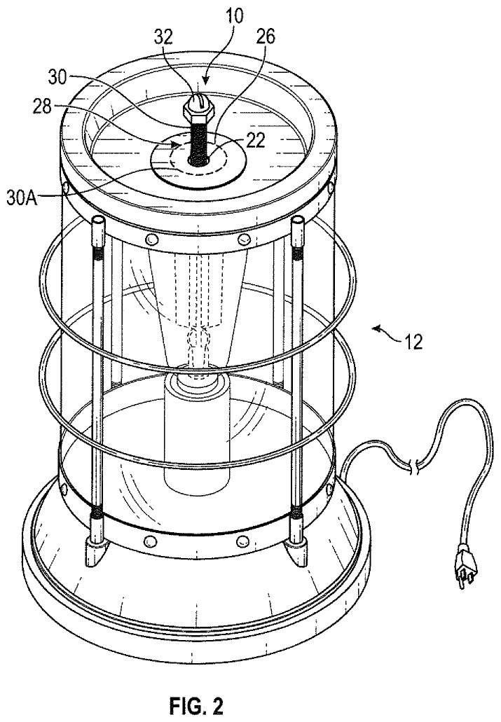

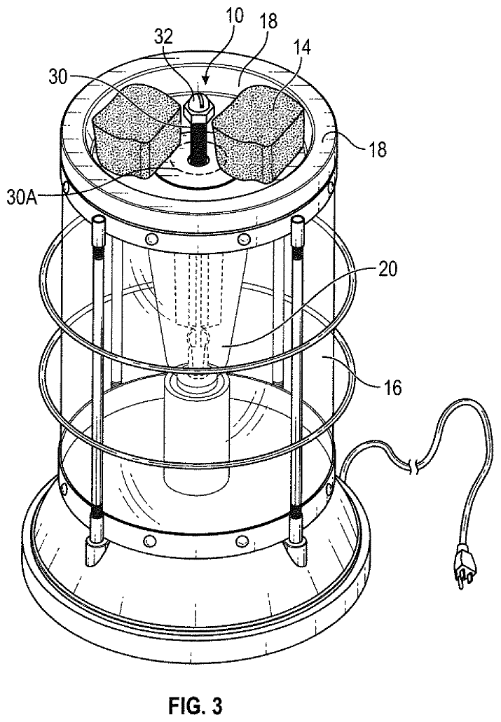

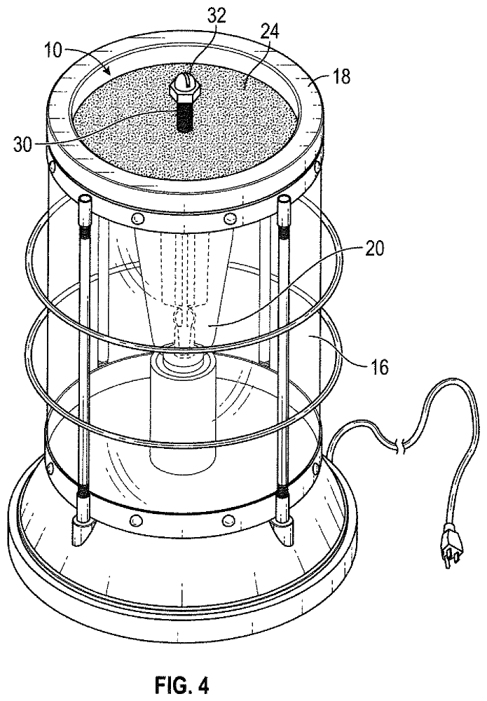

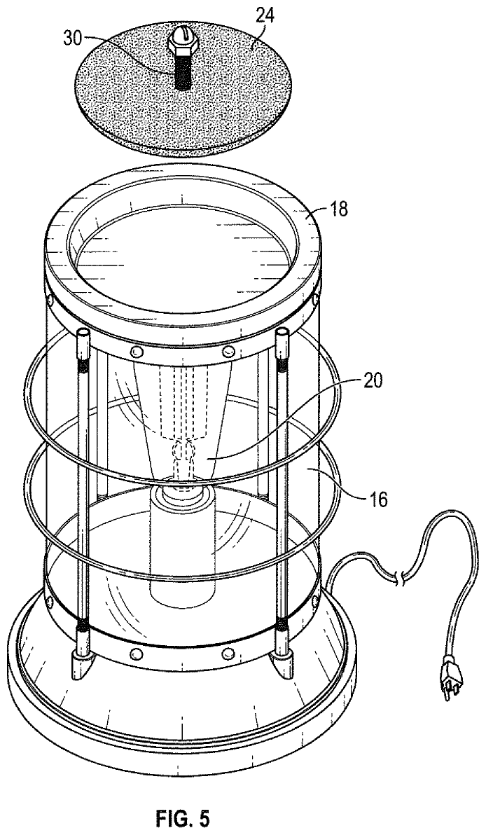

Referring to FIGS. 1-5, a wax removal device 10 (hereinafter device 10) may be shown. The device 10 may be used with a wax heater 12 to remove used wax melts 14 from the wax heater 12. In general, the wax heater 12 may be designed to heat a wax melt 14. As the wax melts 14 are heated, the wax melts 14 release a fragrance or other material contained therein into the surrounding environment. When the wax heater 12 is turned off, the used wax melts 14 harden within the wax heater 12. The device 10 may be used to remove the hardened used wax melts 14.

The wax heater 12 generally includes a body 16. The body 16 may be used to house a heater assembly 20. A reservoir 18 may be placed on a top area of the body 16 and above the heater assembly 20. Wax melts 14 may be placed in the reservoir 18. Wax melts 14 may be small pieces of wax that are like candles in scent and composition. When the heating assembly 20 is turned on, heat from the heating assembly 20 may warm the wax melts 14. As the wax melts 14 are heated, the wax melts 14 begin to melt and may release a pleasant aroma. The melted wax of the wax melts 14 may build up over time within the reservoir 18 and may need to be removed.

The device 10 may be used to remove the melted wax of the wax melts 14. The device 10 may be placed within the reservoir 18 prior to when the wax melts 14 begin to melt. In general, the device 10 may be placed in a center area of the reservoir 18 prior to the wax melts 14 being placed in the reservoir 18. After the wax melts 14 have melted, the heating assembly 20 turned off, and the melted wax of the wax melts 14 has solidified around the device 10, the device 10 may be used to remove the solidified wax 24. As a user lifts the device 10 out of the reservoir 18, the solidified wax 24, which has solidified on the device 10, is lifted out from the reservoir 18.

The device 10 may have a plate member 26. The plate 26 may come in different sizes and/or shapes. In general, the plate member 26 should be sized and shaped to fit within the reservoir 18. In the present embodiment, the plate member 26 may be similar in shape to the reservoir 18. Thus, in this embodiment, the plate member 26 may be circular in shape and sized to fit within a center area of the reservoir 18. However, this is one example and the plate member 26 may come in other geometrical configurations.

Extending up from a center area of the plate member 26 may be a handle 28. The handle 28 may be used to lift the plate 26 and hence the device 10 out of the reservoir 18. The handle 28 may be formed of a rod member 30. The rod member 30 may have a first end coupled to the plate member 16. A distal end of the rod member 30 may have a knob 32 coupled thereto.

In accordance with one embodiment, the plate member 26 may have an opening 22 formed through a center section thereof. Thus, the plate member 26 may be like a ring and/or washer. In this embodiment, the rod member 30 may have a head 30A formed at the proximate end thereof. The head 30A may have a perimeter which is sized larger than the perimeter of the opening 22. This may prevent the head 30A from slipping through the opening 22. The head 30A may raise a center area of the plate member 36 off of the reservoir 18 when the device 10 is positioned within the reservoir 18. The knob 32 is still attached to the distal end of the rod member 30.

In operation, the device 10 is placed in a middle of the reservoir 18 of the wax heater 12 to be used. Wax melts 14 may then be placed in the reservoir 18. It does not make a difference where the wax melts 14 are placed in the reservoir 18 or if the wax melts 14 are in contact with the device 10. When the wax heater 12 is activated, the heating assembly 20 may warm the wax melts 14. As the wax melts 14 are heated, the wax melts 14 begin to melt. When a change of wax melts 14 is desired, the heating assembly 20 may be turned off and the melted wax of the wax melts 14 to be changed may cool and solidified around the device 10. The device 10 may then be used to remove the solidified wax 24. The user may grab the handle 28 of the device 10 and gently wiggle the device 10 back and forth to loosen the solidified wax 24 from the reservoir 18. Once loosened, the user may lift the handle 28 up. The solidified wax 24 may come out in a single piece from the reservoir 18. The user may then dispose of the solidified wax 24. In accordance with one embodiment, the user may break the solidified wax 24 off the device 10. The user may place his/her thumbs on the solidified wax 24 and press inwards towards the plate member 26 of the device 10. The pressure applied should break the solidified wax 24 in half and off the device 10. The broken solidified wax 24 may then be disposed in the garbage. The device 10 may then be placed back in the reservoir 18 for future use.

The foregoing description is illustrative of particular embodiments of the application but is not meant to be a limitation upon the practice thereof. The following claims, including all equivalents thereof, are intended to define the scope of the application.

What is claimed is:

1. A wax removing device for a wax heater comprising:
a plate member sized to fit within a reservoir of the wax heater, and
a handle extending up from the plate member.

2. The wax removing device of claim 1, wherein the handle comprises:
a rod member attached to the plate member; and
a knob attached to a distal end of the rod member.

3. The wax removing device of claim 1, comprising an opening formed within a center area of the plate member.

4. The wax removing device of claim 3, wherein the handle comprises:
a rod member;
a head formed on a first end of the rod member, wherein the head has a larger perimeter than a perimeter of the opening.

5. The wax removing device of claim 4, wherein the handle comprises a knob attached to a distal end of the rod.

6. The wax removing device of claim 4, wherein the head raises the center area of the plate member off of the reservoir when the device is placed within the reservoir of the wax heater.

7. A wax removing device for a wax heater comprising:
a circular plate member sized to fit within a reservoir of the wax heater; and
a handle extending up from a center area of the plate member, wherein the handle comprises:
a rod member attached to the plate member; and
a knob attached to a distal end of the rod member.

8. The wax removing device of claim 7, comprising an opening formed within a center area of the plate member.

9. The wax removing device of claim 8, wherein the handle comprises:
a rod member,
a head formed on a first end of the rod member, wherein the head has a larger perimeter than a perimeter of the opening.

10. The wax removing device of claim 9, wherein the handle comprises a knob attached to a distal end of the rod.

11. The wax removing device of claim 9, wherein the head raises the center area of the plate member off of the reservoir when the device is placed within the reservoir of the wax heater.
